(19) United States
(12) Patent Application Publication
(10) Pub. No.: US 2019/0070597 A1
(43) Pub. Date: Mar. 7, 2019

(54) SYNTHESIS OF PLATE-LIKE SAPO-34 CRYSTALS

(71) Applicant: SABIC Global Technologies B.V., Bergen op Zoom (NL)

(72) Inventors: Dustin Fickel, Sugar Land, TX (US); Neeta Kulkarni, Sugar Land, TX (US)

(21) Appl. No.: 15/767,192

(22) PCT Filed: Sep. 20, 2016
(86) PCT No.: PCT/US2016/052639

§ 371 Date: Apr. 10, 2018

Related U.S. Application Data
(60) Provisional application No. 62/240,749, filed on Oct. 13, 2015.

(51) Int. Cl. B01J 29/85; B01J 37/10; B01J 35/02; C07C 1/30

(52) U.S. Cl. CPC B01J 29/85; B01J 37/10; C07C 2529/85; C07C 1/30; B01J 35/023

(57) ABSTRACT

Disclosed is a SAPO-34 molecular sieve having platelet morphology with the smallest dimension on the order of a few nanometers. Also disclosed are methods and systems of using said molecular sieve for catalyzing the reaction of alkyl halides to light olefins. These methods and systems have been shown to have maximum combined selectivity of ethylene and propylene of at least 90% or ranging from 90% to 98%.

### CROSS REFERENCE TO RELATED APPLICATIONS

This application claims the benefit of priority of U.S. Provisional Patent Application No. 62/240,749, filed Oct. 13, 2015, which is hereby incorporated by reference in its entirety.

### BACKGROUND OF THE INVENTION

### A. Field of the Invention

The invention generally concerns the use of silicoaluminophosphate (SAPO) molecular sieve catalysts to catalyze the reaction of alkyl halides or alcohols to light olefins. In particular, a SAPO-34 catalyst prepared by a hydrothermal method provides a catalyst having plate-like crystal morphology with the smallest dimension on the order of a few nanometers. This catalyst shows higher activity and a longer catalyst lifetime when compared to conventional SAPO-34 catalysts having larger cubic or spherical crystal morphology. Further, the nano-platelet catalysts of the present invention provide good conversion of alkyl halides and selectivity for C₂-C₄ olefins.

### B. Description of Related Art

Light olefins such as ethylene and propylene are used by the petrochemical industry to produce a variety of key chemicals that are then used to make numerous downstream products. By way of example, both of these olefins are used to make a multitude of plastic products that are incorporated into many articles manufacture. FIGS. 1A and 1B provide examples of products generated from ethylene (FIG. 1A) and propylene (FIG. 1B).

Methane activation to higher hydrocarbons, especially to light olefins, has been the subject of great interest over many decades. Recently, the conversion of methane to light olefins via a two-step process that includes conversion of methane to methyl halide, particularly to methyl mono-halide, for example, to methyl chloride followed by conversion of the halide to light olefins as well as the direct methanol to olefin (MTO) reaction has attracted great attention. Micro pore zeolite (e.g., ZSM-5) or zeolite type catalysts (e.g., SAPO-34) have been commonly employed for these methyl chloride (or other methyl halide) and methanol conversion reactions. However, the selectivity to a desired olefin (e.g., propylene), rapid catalyst deactivation due to carbon deposition (coking), and the synthesis cost of the catalyst remain the major challenges for scale-up and commercial success of the reaction.

The size and morphology of catalyst particles/crystals is an important parameter in many catalytic processes. This is especially true for catalytic process where the mean free path/length for the reactant and products plays an important role in the activity and deactivation. For reactions that take place within zeolitic materials, the reactant(s) must diffuse into the crystal and the product(s) must diffuse out by a process called intraparticle diffusion. In reactions where this is important, i.e. methanol to olefins and chloromethane to olefins, it is essential to reduce the diffusion path or mean free path as much as possible in order to ensure good selectivity to target products and to minimize secondary product formation, i.e. coking and deactivation. The materials used and protocols followed for the preparation of SAPO-34 molecular sieves can influence crystal size, crystal morphology, surface Brønsted acidity, and ultimately the overall activity and stability of the final catalysts. For instance, any of the silicon source, structuring directing agents, crystallization conditions, and material composition in initial gel formation, alone or in combination can dramatically influence the average crystal size of the catalyst (See, for example, Razavian et al. in “Recent Advances in Silicoaluminophosphate Nanocatalysts Synthesis Techniques and Their Effects on Particle Size Distribution, Reviews on Advancement of Material Science, 2011, Vol. 29, pp. 83-99). SAPO-34 catalysts have been prepared using hydrothermal methods. By way of example, Chinese patent publications CN102616810B and CN103641131B to Yu et al. and CN103964456 by Xuchen et al. all concern the methanol to olefin (MTO) reaction and the design of SAPO-34 catalysts specific to this reaction. The MTO reaction process requires the presence of an alcohol (e.g., methanol) in the feed stream and involves a cascade of reactions performed under acidic conditions. Catalyst choice, topology and acidity, as well as specific process conditions determine the overall MTO activity and selectivity. MTO reactions are comparable to alkyl halide to olefin (e.g., chloromethane to olefin CTO) reactions in that they both catalyse the formation of olefins from activated methane. However, the overall reaction mechanisms of these reactions in the presence of SAPO-34 catalysts are not fully understood, and different products, activities, and selectivities can be realized from the different starting materials using the same catalyst. For example, in addition to olefins, CTO reactions can produce aromatic compounds and hydrogen chloride, while MTO reactions can produce ethers (e.g., dimethyl ether). (See, for example, Wei et al., Chinese Journal of Catalysis, 2012, 33:11-21). Without wishing to be bound by theory, it is believe that the difference in the two reactions is due to the difference in electron affinity between methanol and chloromethane and the catalyst surface. Thus, when MTO catalysts are used in CTO reactions, lower activity is generally realized.

### SUMMARY OF THE INVENTION

A discovery has been made that solves issues with activity of silicoaluminophosphate (SAPO) molecular sieve catalysts used in alkyl halide to light olefin (e.g., C₂-C₄ olefins) reaction processes. The discovery is premised on a SAPO-34 catalyst prepared by a hydrothermal method that provides a catalyst having plate-like crystal morphology with the smallest dimension on the order of a few nanometers (i.e., a nano-platelet). Without wishing to be bound by theory, it is believed that the current nano-platelet crystal morphology reduces intraparticle diffusion in comparison to conventional SAPO-34 catalysts having larger cubic or spherical crystal morphology, thereby providing improved conversion and selectivity in the alky halide to olefin reaction.

In one aspect of the present invention, there is disclosed a silicoaluminophosphate (SAPO)-34 molecular sieve, namely, SiO₂/Al₂O₃/P₂O₅. In a particular aspect, the synthesis molar ratio of SiO₂/Al₂O₃/P₂O₅ can be 0.6:1:1. The SAPO-34 molecular sieve can have a platelet morphology and a platelet thickness of less than 20 nm. Without wishing to be bound by theory, it is believed that the use of the templating agent tetraethylammonium hydroxide (TEAOH) under specific hydrothermal conditions results in crystals have platelet morphology with the smallest dimension being less than 20 nm. In a particular aspect, the SAPO-34 molecular sieve does not have a spherical morphology, a flower morphology, or a cubic morphology. In some aspects, the SAPO-34 molecular sieve of the current invention is capable of catalytically converting an alkyl halide to an olefin with greater alkyl halide conversion as compared to a SAPO-34 molecular sieve having 0.2 to 4 micron cubic particles, wherein the initial alkyl halide conversion is least 80%.

Also disclosed are methods for converting an alkyl halide to an olefin. A method can include contacting a SAPO-34 catalyst of the present invention having platelet morphology with a feed that includes an alkyl halide under reaction conditions sufficient to produce an olefin hydrocarbon product that includes C₂-C₄ olefins. The alkyl halide used in the method can be a methyl halide (e.g., methyl chloride, methyl bromide, methyl fluoride, or methyl iodide, or any combination thereof). In a particular aspect, the molecular sieve of the current invention has greater alkyl halide conversion as compared to a SAPO-34 molecular sieve having 0.2 to 4 micron cubic particles. The maximum combined selectivity of ethylene and propylene is at least 70%, preferably at least 80%, or more preferably 90% to 98%, wherein the maximum combined space time yield of ethylene and propylene is at least 1/hr or 1/hr to 3/hr, and/or wherein the maximum conversion of alkyl halide is at least 65% or 70% to 80%, wherein the maximum selectivity of ethylene is 50% to 60% and the maximum selectivity of propylene is 35% to 45%. In certain aspects of the method, the reaction for converting an alkyl halide to an olefin occurs in a fluid catalytic cracking (FCC) process or reactor or fluidized circulating bed process or reactor. Reaction conditions for the conversion to an alkyl halide can include a temperature from 300° C. to 500° C., a pressure of 5 atm or less, and a weighted hourly space velocity (WHSV) of 0.5 to 10 h⁻¹, preferably a temperature of 450° C., a pressure of 0.013 MPa, and a WHSV of 3 h⁻¹. The method can also involve collecting or storing the produced olefin hydrocarbon product and using the produced olefin hydrocarbon product to produce a petrochemical or a polymer.

In another embodiment, there is disclosed a method for preparing a silicoaluminophosphate (SAPO)-34 molecular sieve. The method can include; (a) obtaining a mixture comprising water, an aluminum source, a silicon source, a phosphorous source, and a templating agent (e.g., a quaternary ammonium salt); (b) treating the mixture to a temperature of 130° C. to 190° C., preferably 150° C. to 170° C. under autogenous pressure to obtain a crystalline material; and (c) removing the templating agent from the crystalline material to obtain the SAPO-34 molecular sieve having platelet morphology and the thickness of the platelet less than 20 nm. In one aspect of the method, the mixture in (a) has a molar composition of:

aR:bSiO₂:cAl₂O₃:dP₂O₅:eH₂O,

where R is the templating agent, and a is 0-1, b is 0-1, c is 0-1, d is 0-1, and e is 30 to 80. In one instance a is 1, b is 0.65, c is 1, d is 1, and e is 45 and the aluminum source is aluminum isopropoxide, the phosphorous source is phosphoric acid, and the silicon source is colloidal silica, fumed silica, or tetraethyl orthosilicate. The templating agent can include tetraethylammonium hydroxide, and in one particular aspect the templating agent consists or consists essentially of tetraethylammonium hydroxide. In another aspect, step (b) can be performed at a temperature of 120° C. to 180° C., preferably 140° C. to 160° C. for 48 hours to 196 hours, preferably 96 hours to 144 hours. Step (c) can include subjecting the crystalline material to a temperature of 400° C. to 700° C., preferably 500° C. to 600° C. for a period of 2 hours to 12 hours, preferably 3 hours to 10 hours.

In still another embodiment of the present invention, there is disclosed a system for producing olefins. The system can include an inlet for a feed including the alkyl halide discussed above and throughout this specification, a reaction zone that is configured to be in fluid communication with the inlet, and an outlet configured to be in fluid communication with the reaction zone to remove an olefin hydrocarbon product from the reaction zone. The reaction zone can include the silicoaluminophosphate (SAPO)-34 molecular sieve of the present invention, the feed and the olefin hydrocarbon product. In some instances, the olefin hydrocarbon product can include ethylene, propylene, or both. The system of the current embodiments can further include a collection device that is capable of collecting the olefin hydrocarbon product.

Also disclosed in the context of the present invention are embodiments 1-33. Embodiment 1 is a silicoaluminophosphate (SAPO)-34 molecular sieve comprising SiO₂/Al₂O₃/P₂O₅, wherein the SAPO-34 molecular sieve has platelet morphology and the thickness of the platelet is less than 20 nm. Embodiment 2 is the SAPO-34 molecular sieve of embodiment 1, wherein the synthesis molar ratio of SiO₂/Al₂O₃/P₂O₅ is 0.6:1:1. Embodiment 3 is the SAPO-34 molecular sieve of any one of embodiments 1 to 2, wherein the SAPO-34 molecular sieve has been templated from a tetraethylammonium hydroxide (TEAOH) under hydrothermal conditions. Embodiment 4 is the SAPO-34 molecular sieve of any one of embodiments 1 to 3, wherein the SAPO-34 molecular sieve does not have a spherical morphology, a flower morphology, or a cubic morphology. Embodiment 5 is the SAPO-34 molecular sieve of any one of embodiments 1 to 4, wherein the SAPO-34 molecular sieve is capable of converting an alkyl halide to an olefin. Embodiment 6 is the SAPO-34 molecular sieve of embodiment 5, wherein the SAPO-34 molecular sieve has greater alkyl halide conversion as compared to 0.2 to 4 micron SAPO-34 molecular sieve cubic particles. Embodiment 7 is the SAPO-34 molecular sieve of embodiment 6, wherein the SAPO-34 molecular sieve initial alkyl halide conversion is least 80%.

Embodiment 8 is a method for converting an alkyl halide to an olefin, the method comprising contacting a SAPO-34 catalyst with a feed comprising an alkyl halide under reaction conditions sufficient to produce an olefin hydrocarbon product comprising C₂-C₄ olefins, wherein the SAPO-34 molecular sieve catalyst has a platelet morphology and has been templated under hydrothermal conditions from a mixture comprising a quaternary ammonium salt. Embodiment 9 is the method of embodiment 8, wherein the quaternary ammonium salt is tetraethylammonium hydroxide (TEOH). Embodiment 10 is the method of any one of embodiments 8 to 9, wherein the smallest dimension of the platelet is 20 nm. Embodiment 11 is the method of any one of embodiments 8 to 10, wherein the alkyl halide is a methyl halide. Embodiment 12 is the method of embodiment 11, wherein the methyl halide is methyl chloride, methyl bromide, methyl fluoride, or methyl iodide, or any combination thereof. Embodiment 13 is the method of any one of embodiments 8 to 12, wherein the molecular sieve has greater alkyl halide conversion as compared to 0.2 to 4 micron SAPO-34 molecular sieve cubic particles. Embodiment 14 is the method of embodiment 13, wherein the maximum combined selectivity of ethylene and propylene is at least 70%, preferably at least 80%, or more preferably 90% to 98%, wherein the maximum combined space time yield of ethylene and propylene is at least 1/hr or 1/hr to 3/hr, and/or wherein the maximum conversion of alkyl halide is at least 65% or 70% to 80%. Embodiment 15 is the method of embodiment 14, wherein the maximum selectivity of ethylene is 50% to 60% and the maximum selectivity of propylene is 35% to 45%. Embodiment 16 is the method of any one of embodiments 8 to 15, wherein the reaction occurs in a fluid catalytic cracking (FCC) reactor or fluidized circulating bed reactor. Embodiment 17 is the method of any one of embodiments 8 to 16, wherein the reaction conditions include a temperature from 300° C. to 500° C., a pressure of 5 atm or less, and a weighted hourly space velocity (WHSV) of 0.5 to 10 h⁻¹, preferably a temperature of 450° C., a pressure of 0.013 MPa, and a WHSV of 3 h⁻¹. Embodiment 18 is the method of any one of embodiments 8 to 17, further comprising collecting or storing the produced olefin hydrocarbon product. Embodiment 19 is the method of any one of embodiments 8 to 18, further comprising using the produced olefin hydrocarbon product to produce a petrochemical or a polymer. Embodiment 20 is a method for preparing a silicoaluminophosphate (SAPO)-34 molecular sieve of embodiments 1 to 7, the method comprising: (a) obtaining a mixture comprising water, an aluminum source, a silicon source, a phosphorous source, and a templating agent, wherein the templating agent comprises a quaternary ammonium salt; (b) treating the mixture to a temperature of 150° C. to 170° C. under autogenous pressure to obtain a crystalline material; and (c) removing the templating agent from the crystalline material to obtain the SAPO-34 molecular sieve. Embodiment 21 is the method of embodiment 20, wherein the mixture in (a) has a molar composition of:

aR:bSiO₂:cAl₂O₃:dP₂O₅:eH₂O,

where R is the templating agent, and a is 0-1, b is 0-1, c is 0-1, d is 0-1, and e is 30 to 80. Embodiment 22 is the method of embodiment 21, wherein a is 1, b is 0.65, c is 1, d is 1, and e is 45. Embodiment 23 is the method of any one of embodiments 20 to 22, wherein the aluminum source is aluminum isopropoxide, the phosphorous source is phosphoric acid, and the silicon source is colloidal silica, fumed silica, or tetraethyl orthosilicate. Embodiment 24 is the method of any one of embodiments 20 to 23, wherein the templating agent further comprises a quaternary ammonium salt. Embodiment 25 is the method of any one of embodiments 20 to 24, wherein the quaternary ammonium salt is tetraethylammonium hydroxide. Embodiment 26 is the method of embodiment 25, wherein the templating agent consists or consists essentially of tetraethylammonium hydroxide. Embodiment 27 is the method of any one of embodiments 20 to 26, wherein step (b) is performed at a temperature of 140° C. to 160° C. for 96 hours to 144 hours. Embodiment 28 is the method of any one of embodiments 20 to 27, wherein step (c) comprises subjecting the crystalline material to a temperature of 500° C. to 600° C. for 3 hours to 10 hours. Embodiment 29 is a method for converting an alkyl halide to an olefin, the method comprising contacting a SAPO-34 molecular sieve of any one of embodiments 1 to 7 with a feed comprising an alkyl halide under reaction conditions sufficient to produce an olefin hydrocarbon product comprising C₂-C₄ olefins.

Embodiment 30 is a system for producing olefins, the system comprising: an inlet for a feed comprising an alkyl halide; a reaction zone that is configured to be in fluid communication with the inlet, wherein the reaction zone comprises the catalysts of any one of embodiments 1 to 7; and an outlet configured to be in fluid communication with the reaction zone to remove an olefin hydrocarbon product from the reaction zone. Embodiment 31 is the system of embodiment 30, wherein the reaction zone further comprises the feed and the olefin hydrocarbon product. Embodiment 32 is the system of embodiment 31, wherein the olefin hydrocarbon product comprises ethylene and propylene. Embodiment 32 is the system of any one of embodiments 30 to 32, further comprising a collection device that is capable of collecting the olefin hydrocarbon product.

The following includes definitions of various terms and phrases used throughout this specification.

The term “catalyst” means a substance which alters the rate of a chemical reaction. “Catalytic” means having the properties of a catalyst.

The term “conversion” means the mole fraction (i.e., percent) of a reactant converted to a product or products.

The term “selectivity” refers to the percent of converted reactant that went to a specified product, for example C₂-C₄ olefin selectivity is the % of alkyl halide that formed C₂-C₄ olefins.

The term “platelet” refers to a plate-like or tabular shape having dimensions of length, width, and thickness. A “nano-platelet” in the context of this application refers to crystal having platelet morphology in which at least one dimension of the crystal is equal to or less than 20 nm (e.g., one dimension is 1 to 20 nm in size). A nano-platelet can have at least two dimensions that are equal to or less than 20 nm (e.g., a first dimension is 1 to 20 nm in size and a second dimension is 1 to 20 nm in size).

The term “template” means any synthetic and/or natural material that provides at least one nucleation site where ions can nucleate and grow to form crystalline material.

The term “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The catalysts of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the catalysts of the present invention are their ability to selectivity produce light olefins, and in particular, ethylene and propylene, from alkyl halides (e.g., methyl chloride).

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIGS. 1A and 1B depict illustrations of various chemicals and products that can be produced from ethylene (FIG. 1A) and propylene (FIG. 1B).

FIG. 2 depicts a system for producing olefins from alkyl halides using the catalyst of the present invention.

FIG. 3 shows scanning transmission electron microscopy (STEM) images of the SAPO-34 molecular sieve of the current invention at 2000×, 5000×, 10000×, and 30000× magnifications.

FIG. 4 shows scanning transmission electron microscopy (STEM) images of a conventionally prepared SAPO-34 molecular sieve at 10000× and 30000× magnifications.

FIG. 5 shows graphical data for chloromethane conversion (y axis) and time of stream (x axis) comparing the SAPO-34 molecular sieve of the current invention with a conventionally prepared SAPO-34 molecular sieve.

### DETAILED DESCRIPTION OF THE INVENTION

SAPO catalysts have an open microporous structure with regularly sized channels, pores or “cages.” These materials are sometimes referred to as “molecular sieves” in that they have the ability to sort molecules or ions based primarily on the size of the molecules or ions. SAPO materials are both microporous and crystalline and have a three-dimensional crystal framework of PO₄⁺, AlO₄⁻ and SiO₄ tetrahedra. Among developed catalysts, silicoaluminophosphate SAPO-34 molecular sieve having 8-membered ring pore sizes of 0.43-0.50 nm, relatively mild acidity, and good thermal/hydrothermal stability, is recognized as a good catalyst for methanol to olefin (MTO) reactions. The catalysts, however, when used in an alkyl halide to olefin reaction suffer from low activity. These MTO and alkyl halide to olefin catalysts have cubic or spherical crystal morphology, which can limit the diffusion of the molecules out of the catalyst. Without wishing to be bound by theory, it is believe that slow intraparticle diffusion can lead to polymerization or homologation of the light olefins, coking of the catalyst, and/or deactivation of the catalyst.

A discovery has been made to produce a SAPO-34 catalyst with small dimensions that aid in minimizing the diffusion path of the reactants and products in and out of the crystal, respectively. Such a discovery is premised on a SAPA-34 molecular sieve (e.g., SiO₂/Al₂O₃/P₂O₅) having a platelet morphology with a thickness less than 20 nm. Without wishing to be bound by theory, it is believed that such properties of the catalyst reduce the diffusion path. Furthermore, methods and systems for using the nano-platelet SAPO-34 molecular sieve for the production of C₂-C₄ olefins from alkyl halides are also disclosed herein. Using these methods and systems, the maximum combined selectivity of ethylene and propylene is at least 70%, preferably at least 80%, or more preferably 90% to 98%.

These and other non-limiting aspects of the present invention are discussed in further detail in the following sections with reference to the Figures.

### A. Nano-Platelet SAPO-34 Catalysts

The synthesis of SAPO-34 catalysts can involve multiple protocols and subtle changes in the preparative details, which can result in dramatic alteration in the physical and chemical properties of the final catalysts. Crystal morphology can resemble a wire, a particle, a sphere, a rod, a tetrapod, a hyper-branched structure, a tube, a cube, a plate, or mixtures thereof. The SAPO-34 catalysts of the present invention are prepared through an alteration of the synthetic recipe and parameters followed by hydrothermal conditions to crystallize the product catalyst. The result is an unexpected crystalline product having nano-platelet morphology with the smallest dimension of less than 20 nm. The nanoscale dimensions less than 20 nm include all dimensions between 0.1 and 20 nm, for instance 1, nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, or 19 nm, and all values in between. The crystals of the prepared SAPO-34 catalyst do not have a spherical, flower, or cubic morphology.

Generally, the SAPO-34 catalysts are prepared using a gel containing aluminum (Al), phosphorus (P) and silicon (Si) compounds with structure-directing agents under crystallization conditions. Conventionally SAPO-34 catalysts are formed as 0.2 to 4 micron cubic particles. In one embodiment, the SAPO-34 catalysts with nano-platelet crystal morphology are prepared with SiO₂, Al₂O₃, and P₂O₅. The synthesis molar ratio of SiO₂/Al₂O₃/P₂O₅ can be 0.4:1:1, 0.45:1:1, 0:5:1:1, 0.55:1:1, 0.6:1:1, 0.65:1:1, 0.7:1:1, 0.75:1:1, 0.8:1:1, 0.6:0.8:1, 0.6:0.85, 0.6:0.9:1, 0.6:0.95:1, 0.6:1.1:1, 0.6:1.15:1, 0.6:1.2:1, 0.6:1:0.8, 0.6:1:0.85, 0.6:1:0.9, 0.6:1:0.95, 0.6:1:1.05, 0.6:1:1.1, 0.6:1:1.15, or 0.6:1:1.2. Preferably, the synthesis molar ratio of SiO₂/Al₂O₃/P₂O₅ is 0.6:1:1, templating with tetraethylammonium hydroxide (TEAOH). In coordination chemistry, a template reaction is a ligand-based reaction that occurs between two or more adjacent coordination sites on a metal center. The addition of a structure-directing or template agent/ion effects the pre-organization provided by the coordination sphere and can results in significant modification of physical/chemical/electronic properties of the template complex formed. Examples of templating agents include organic amines such as tetraethylammonium hydroxide (TEAOH).

Non-limiting examples of making SAPO catalysts of the present invention are provided in the Examples section. Method of making the SAPO catalysts can include one or more steps that can be used in any order. By way of example, an aqueous mixture of an aluminum source, a silicon source, a phosphorous source, and a templating agent, (e.g., a quaternary ammonium salt) can be obtained. The aqueous mixture can be heated to a temperature of 130° C. to 190° C., preferably 150° C. to 170° C. under autogenous pressure to obtain a crystalline material. The mixture can be cooled, and the templating agent can be removed from the crystalline material to obtain the SAPO-34 molecular sieve having platelet morphology and the thickness of the platelet less than 20 nm. The synthesis mixture can have a general structure:

aR:bSiO₂:cAl₂O₃:dP₂O₅:eH₂O,

where R is the templating agent, and a is 0-1, b is 0-1, c is 0-1, d is 0-1, and e is 30 to 80. Specifically, the ratios of reactants can be a is 0.8, b is 0.65, c is 1, d is 1, and e is 40-50; a is 0.85, b is 0.65, c is 1, d is 1, and e is 40-50; a is 0.9, b is 0.65, c is 1, d is 1, and e is 40-50; a is 0.95, b is 0.65, c is 1, d is 1, and e is 40-50; a is 1, b is 0.65, c is 1, d is 1, and e is 40-50; a is 1.05, b is 0.65, c is 1, d is 1, and e is 40-50; a is 1.1, b is 0.65, c is 1, d is 1, and e is 40-50; a is 1.15, b is 0.65, c is 1, d is 1, and e is 40-50; a is 1.2, b is 0.65, c is 1, d is 1, and e is 40-50; a is 0.8, b is 0.55, c is 1, d is 1, and e is 40-50; a is 0.8, b is 0.6, c is 1, d is 1, and e is 40-50; a is 0.8, b is 0.7, c is 1, d is 1, and e is 40-50; a is 0.8, b is 0.75, c is 1, d is 1, and e is 40-50; a is 0.8, b is 0.8, c is 1, d is 1, and e is 40-50; a is 0.8, b is 0.65, c is 0.8, d is 1, and e is 40-50; a is 0.8, b is 0.65, c is 0.85, d is 1, and e is 40-50; a is 0.8, b is 0.65, c is 0.9, d is 1, and e is 40-50; a is 0.8, b is 0.65, c is 0.9, d is 1, and e is 40-50; a is 0.8, b is 0.65, c is 0.95, d is 1, and e is 40-50; a is 0.8, b is 0.65, c is 1.05, d is 1, and e is 40-50; a is 0.8, b is 0.65, c is 1.1, d is 1, and e is 40-50; a is 0.8, b is 0.65, c is 1.15, d is 1, and e is 40-50; a is 0.8, b is 0.65, c is 1.2, d is 1, and e is 40-50; a is 0.8, b is 0.65, c is 1, d is 0.8, and e is 40-50; a is 0.8, b is 0.65, c is 1, d is 0.85, and e is 40-50; a is 0.8, b is 0.65, c is 1, d is 0.9, and e is 40-50; a is 0.8, b is 0.65, c is 1, d is 0.95, and e is 40-50; a is 0.8, b is 0.65, c is 1, d is 1.05, and e is 40-50; a is 0.8, b is 0.65, c is 1, d is 1.1, and e is 40-50; a is 0.8, b is 0.65, c is 1, d is 1.15, and e is 40-50; or a is 0.8, b is 0.65, c is 1, d is 1.2, and e is 40-50. In one instance, a is 1, b is 0.65, c is 1, d is 1, and e is 45. The aluminum source can be aluminium methoxide, aluminium ethoxide, aluminum isopropoxide, or aluminium tert-butoxide. The phosphorus source can be phosphoric acid. The silicon source can be colloidal silica, fumed silica, tetramethyl orthosilicate, tetraethyl orthosilicate, or tetraisopropyl orthosilicate. A non-limiting example, of a commercial source of the above mentioned aluminum, phosphorus, and silicon sources is Sigma Aldrich® (U.S.A).

In a particular aspect, the materials having nano-platelet morphology in the current invention are prepared by a hydrothermal processes. Hydrothermal processes can include techniques of crystallizing the material from high-temperature aqueous solutions at high vapor pressures. Crystal growth can be performed in a pressure vessel, such as an autoclave using autogenous pressure, by a temperature-difference method, temperature-reduction method, or a metastable-phase technique. In a particular embodiment, the crystal growth is performed in an autoclave. In one aspect of the hydrothermal process, the aqueous synthesis gel prepared by mixing the reactants can be autoclaved at a temperature of 120° C. to 180° C., preferably 140° C. to 160° C., and all temperatures therebetween including 141° C., 142° C., 143° C., 144° C., 145° C., 146° C., 147° C., 148° C., 149° C., 150° C., 151° C., 152° C., 153° C., 154° C., 155° C., 156° C., 157° C., 158° C., or 159° C., for 48 hours to 196 hours, preferably 96 hours to 144 hours, and all periods of time therebetween including 97 hours, 98 hours, 99 hours, 100 hours, 101 hours, 102 hours, 103 hours, 104 hours, 105 hours, 106 hours, 107 hours, 108 hours, 109 hours, 110 hours, 111 hours, 112 hours, 113 hours, 114 hours, 115 hours, 116 hours, 117 hours, 118 hours, 119 hours, 120 hours, 121 hours, 122 hours, 123 hours, 124 hours, 125 hours, 126 hours, 127 hours, 128 hours, 129 hours, 130 hours, 131 hours, 132 hours, 133 hours, 134 hours, 135 hours, 136 hours, 137 hours, 138 hours, 139 hours, 140 hours, 141 hours, 142 hours, or 143 hours to provide the nano-platelet catalyst. In another aspect, the nano-platelet catalysts can be further calcined. Calcination can include subjecting the crystalline material to a temperature of 400° C. to 700° C., preferably 500° C. to 600° C. and all temperature there between including 510° C., 520° C., 525° C., 530° C., 535° C., 540° C., 545° C., 550° C., 555° C., 560° C., 565° C., 570° C., 575° C., 580° C., 585° C., 590° C., or 595° C., for a period of 2 hours to 12 hours, preferably 3 hours to 10 hours, and all times there between including 3.5 hours, 4 hours, 4.5 hours, 5 hours, 5.5 hours, 6 hours, 6.5 hours, 7 hours, 7.5 hours, 8 hours, 8.5 hours, 9 hours, or 9.5 hours in the presence of an oxygen source (e.g., air).

In a further aspect, a method is disclosed for converting an alkyl halide to an olefin. The method can include contacting a SAPO-34 molecular sieve including SiO₂/Al₂O₃/P₂O₅, where the SAPO-34 molecular sieve has platelet morphology and the thickness of the platelet is less than 20 nm, with a feed containing an alkyl halide under reaction conditions sufficient to produce an olefin hydrocarbon product stream that includes C₂-C₄ olefins.

Without wishing to be bound by theory, it is believed that SAPO-34 catalysts prepared using the specified equivalents of reactants, TEAOH as a templating agent, under the specified hydrothermal conditions surprisingly provides nano-platelet crystal morphology that minimizes the diffusion path through the catalytic material, which benefits the methods and systems for converting alkyl halides to olefins as currently disclosed.

### B. Alkyl Halide Feed

The alkyl halide feed can include one or more alkyl halides. The alkyl halide feed can contain alkyl mono halides, alkyl dihalides, alkyl trihalides, preferably alkyl mono halide with less than 10% of other halides relative to the total halides. The alkyl halide feed can also contain nitrogen, helium, steam, and so on as inert compounds. The alkyl halide in the feed can have the following structure: CₙH₍₂ₙ₊₂₎₋ₘXₘ, where n and m are integers, n ranges from 1 to 5, preferably 1 to 3, even more preferably 1, m ranges 1 to 3, preferably 1, X is Br, F, I, or Cl. In particular aspects, the feed can include about 10, 15, 20, 40, 50 mole % or more of the alkyl halide. In a particular embodiment, the feed can contain up to 10 mole % or more of a methyl halide. In preferred aspects, the methyl halide is methyl chloride, methyl bromide, methyl fluoride, or methyl iodide, or any combination thereof. The feed stream can also include some alcohol. In a particular embodiment, the feed stream includes less than 5 wt. % alcohol, preferably less than 1 wt. % alcohol, or preferably is alcohol free (e.g., less 0.01 wt. %, or 0 wt. % or not detectable alcohol). In one instance, the alcohol is methanol.

The production of alkyl halide, particularly of methyl chloride (CH₃Cl, See Equation (I) below), is commercially produced by thermal chlorination of methane at 400° C. to 450° C. and at a raised pressure. Catalytic oxychlorination of methane to methyl chloride is also known. In addition, methyl chloride is commercially manufactured by reaction of methanol and HCl at 180° C. to 200° C. using a catalyst. Alternatively, methyl halides are commercially available from a wide range of sources (e.g., Praxair, Danbury, Conn.; Sigma-Aldrich Co. LLC, St. Louis, Mo.; BOC Sciences USA, Shirley, N.Y.). In preferred aspects, methyl chloride and methyl bromide can be used alone, or in combination.

### C. Olefin Production

The nano-platelet SAPO-34 catalysts of the present invention catalyze the conversion of alkyl halides to C₂-C₄ olefins such as ethylene, propylene and butenes (e.g., 1-butene and/or 2-butene). The following non-limiting two-step process is an example of conversion of methane to methyl chloride and conversion of methyl chloride to ethylene, propylene and butylene. The second step (Equation (II)) illustrates the reactions that are believed to occur in the context of the present invention:

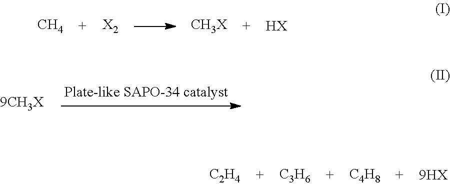

Besides the C₂-C₄ olefins the reaction may produce byproducts such as methane, C₅ olefins, C₂-C₅ alkanes and aromatic compounds such as benzene, toluene and xylene.

Conditions sufficient for olefin production (e.g., ethylene, propylene and butylene as shown in Equation (II)) include temperature, time, alkyl halide concentration, space velocity, and pressure. The temperature range for olefin production may range from about 300° C. to 500° C., preferably ranging 350° C. to 450° C. A weight hourly space velocity (WHSV) of alkyl halide higher than 0.5 h⁻¹ can be used, preferably between 1.0 and 10 h⁻¹, more preferably between 2.0 and 3.5 h⁻¹. The conversion of alkyl halide is carried out at a pressure less than 145 psig (1 MPa) and preferably less than 73 psig (0.5 MPa), or at atmospheric pressure (0.101 MPa). The conditions for olefin production can be varied based on the type of the reactor.

The methods and system disclosed herein can also include the ability to regenerate used/deactivated catalyst in a continuous process such as in a fluid catalytic cracking (FCC)-type process or reactor or a circulating catalyst bed process or reactor. The method and system can further include collecting or storing the produced olefin hydrocarbon product along with using the produced olefin hydrocarbon product to produce a petrochemical or a polymer.

### D. Catalyst Activity/Selectivity

Catalytic activity as measured by alkyl halide conversion can be expressed as the percent (%) moles of the alkyl halide converted with respect to the moles of alkyl halide fed. In particular aspects, the combined selectivity of ethylene and propylene is at least 70%, preferably at least 80%, more preferably at least 90%, or most preferably 90% to 98% (e.g., 90%, 91%, 92%, 93%, 94%, 95%, 96%, 97%, 98% or more) under certain reaction conditions. The maximum combined space time yield (STY) of ethylene and propylene can be at least 1/hr, or 1/hr to 3/hr. The maximum conversion of alkyl halide can be at least 65% or 70% to 80%, 75%, 80%, 90%, or 100%. In certain instances, the selectivity of ethylene is about 40% or higher and the selectivity of propylene is about 30% or higher. The maximum selectivity of ethylene can be 50% to 60% (e.g., 50%, 51%, 52%, 53%, 54%, 55%, 56%, 57%, 58%, 59%, 60%, or more) and the maximum selectivity of propylene is 35% to 45% (e.g., 35%, 36%, 37%, 38%, 39%, 40%, 41%, 42%, 43%, 44%, 45%, or more).

As an example, chloromethane (CH₃Cl) can be used define conversion and maximum selectivity of products by the following equations (III)-(VII):

$$\% {CH}_{3} Cl Conversion = \frac{( {CH}_{3} Cl ) ° - ( {CH}_{3} Cl )}{( {CH}_{3} Cl ) °} \times 100 , \qquad ( III )$$

where, (CH₃Cl)^{o} and (CH₃Cl) are moles of methyl chloride in the feed and reaction product, respectively.

Maximum selectivity is defined as C-mole % and is defined for ethylene, propylene, and so on as follows:

$$\% Ethylene Selectivity = \frac{2 ( {C}_{2} {H}_{4} )}{( {CH}_{3} Cl ) ° - ( {CH}_{3} Cl )} \times 100 , \qquad ( IV )$$

where the numerator is the carbon adjusted mole of ethylene and the denominator is the moles of carbon converted.

Maximum selectivity for propylene may be expressed as:

$$\% Propylene Selectivity = \frac{3 ( {C}_{3} {H}_{6} )}{( {CH}_{3} Cl ) ° - ( {CH}_{3} Cl )} \times 100 , \qquad ( V )$$

where the numerator is the carbon adjusted mole of propylene and the denominator is the moles of carbon converted.

Maximum selectivity for butylene may be expressed as:

$$\% Butylene Selectivity = \frac{4 ( {C}_{4} {H}_{8} )}{( {CH}_{3} Cl ) ° - ( {CH}_{3} Cl )} \times 100 , \qquad ( VI )$$

where the numerator is the carbon adjusted mole of butylene and the denominator is the moles of carbon converted.

Selectivity for aromatic compounds may be expressed as:

$$\% Aromatics Selectivity = \frac{6 ( {C}_{6} {H}_{6} ) + 7 ( {C}_{7} {H}_{8} ) + 8 ( {C}_{8} {H}_{10} )}{( {CH}_{3} Cl ) ° - ( {CH}_{3} Cl )} \times 100 \qquad ( VII )$$

### E. Olefin Production System

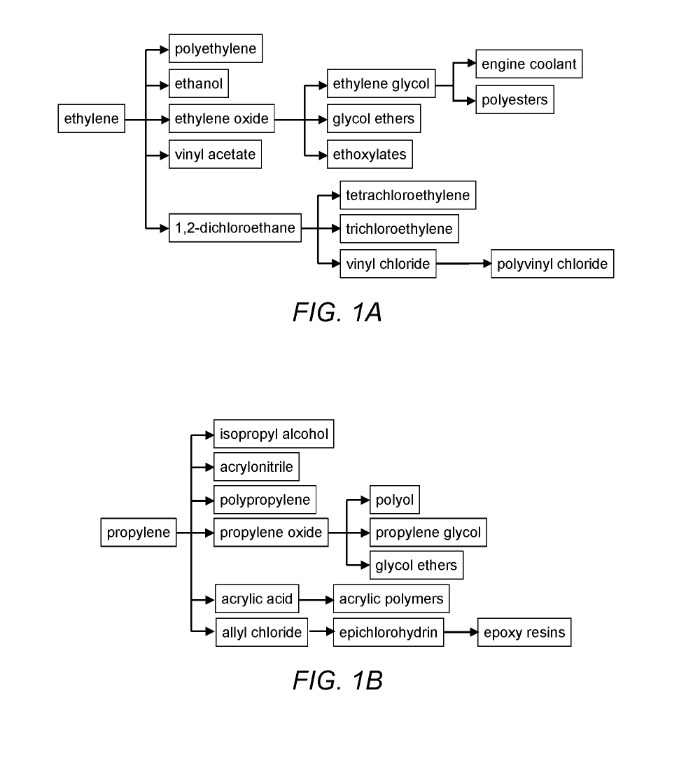

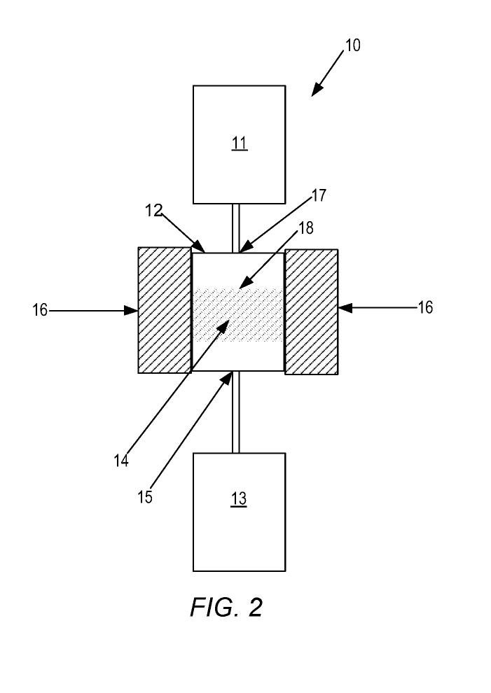

Referring to FIG. 2, a system 10 is illustrated, which can be used to convert alkyl halides to olefin hydrocarbon products with the nano-platelet SAPO-34 catalysts of the present invention. The system 10 can include an alkyl halide source 11, a reactor 12, and a collection device 13. The alkyl halide source 11 can be configured to be in fluid communication with the reactor 12 via an inlet 17 on the reactor. As explained above, the alkyl halide source can be configured such that it regulates the amount of alkyl halide feed entering the reactor 12. The reactor 12 can include a reaction zone 18 having the nano-platelet SAPO-34 catalyst 14 of the present invention. The amounts of the alkyl halide feed 11 and the catalyst 14 used can be modified as desired to achieve a given amount of product produced by the system 10. Non-limiting examples of reactors that can be used include fixed-bed reactors, fluidized bed reactors, bubbling bed reactors, slurry reactors, rotating kiln reactors, or any combinations thereof when two or more reactors are used. Fluid catalytic cracking (FCC)-type reactors or circulating catalyst bed reactors permit the regeneration of used/deactivated catalyst in a continuous process. The reactor 12 can include an outlet 15 for products produced in the reaction zone 18. The products produced can include ethylene, propylene and butylene. The collection device 13 can be in fluid communication with the reactor 12 via the outlet 15. Both the inlet 17 and the outlet 15 can be open and closed as desired. The collection device 13 can be configured to store, further process, or transfer desired reaction products (e.g., C₂-C₄ olefins) for other uses. By way of example only, FIG. 1 provides non-limiting uses of propylene produced from the catalysts and processes of the present invention. Still further, the system 10 can also include a heating source 16. The heating source 16 can be configured to heat the reaction zone 18 to a temperature sufficient (e.g., 325 to 375° C.) to convert the alkyl halides in the alkyl halide feed to olefin hydrocarbon products. A non-limiting example of a heating source 16 can be a temperature controlled furnace. Additionally, any unreacted alkyl halide can be recycled and included in the alkyl halide feed to further maximize the overall conversion of alkyl halide to olefin products. Further, certain products or byproducts such as butylene, C₅₊ olefins and C₂₊ alkanes can be separated and used in other processes to produce commercially valuable chemicals (e.g., propylene). This increases the efficiency and commercial value of the alkyl halide conversion process of the present invention.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results. The materials used in the following examples are described in Table 1, and were used as-described unless specifically stated otherwise.

TABLE 1

| Material | Source |
|---|---|
| Colloidal Silica (Ludox SM-40), 40 wt. % SiO2 | Sigma-Aldrich ® |
| Aluminum iso-propoxide (Al(O-i-Pr)3), ≥98% purity | Sigma-Aldrich ® |
| Phosphoric acid (H3PO4) 85 wt. % aqueous | Sigma Aldrich ® |
| Hydrochloric acid (HCl), 37 wt. % HCl aqueous | Sigma-Aldrich ® |
| Tetraethylammonium hydroxide ((C2H5)4N(OH)), | Sigma Aldrich ® |
| 35 wt. % aqueous | |
| Water (deionized) | SABIC labs |

### Example 1

### Catalyst of the Present Invention Preparation

Catalyst 100 was prepared from the ingredients listed in Table 1 in a synthesis molar ratio of 1TEAOH:0.6SiO₂:1Al₂O₃:1P₂O₅:45H₂O. Aluminum isopropoxide was added slowly (over 30 min) with vigorous stirring to avoid formation of lumps to an aqueous solution of H₃PO₄. The slurry was allowed to stir for 2.5 hrs. Next, colloidal silica was added drop-wise (over 15 min) to the slurry and the resulting mixture was stirred for 1 hour. TEAOH was then added to the above slurry and the mixture was stirred for 2.5 hours. The synthesis gel was transferred into a Teflon liner and placed inside a stainless-steel autoclave and was heated at 150° C. for 120 hours. The solid product was separated by centrifugation, washed with distilled water several times, and dried overnight at 90° C. The dried catalyst sample was calcined at 550° C. for 3-10 h to remove any remaining organic template and powdered.

### Example 2

### Comparative Catalyst Preparation

Comparative catalyst 200, was prepared from the ingredients listed in Table 1 in a synthesis molar ratio of 1.2TEAOH:0.3SiO₂:1Al₂O₃:1P₂O₅:57H₂O and using conventional SAPO-34 methodology. Aluminum isopropoxide was added slowly (over 30 min) with vigorous stirring to avoid formation of lumps to an aqueous solution of H₃PO₄. The slurry was allowed to stir for 2.5 hrs. Next, colloidal silica was added drop-wise (over 15 min) to the slurry and the resulting mixture was stirred for 1 hour. TEAOH was then added to the above slurry and the mixture was stirred for 2.5 hours. The synthesis gel was transferred into a Teflon liner and placed inside a stainless-steel autoclave and was heated at 190° C. for 24 hours. The solid product was separated by centrifugation, washed with distilled water several times, and dried overnight at 90° C. The dried catalyst sample was calcined at 550° C. for 3-10 h to remove any remaining organic template and powdered.

### Example 3

### Characterization of Catalyst 100 and Comparative Catalyst 200

Catalyst 100, Nano-Platelet SAPO-34:

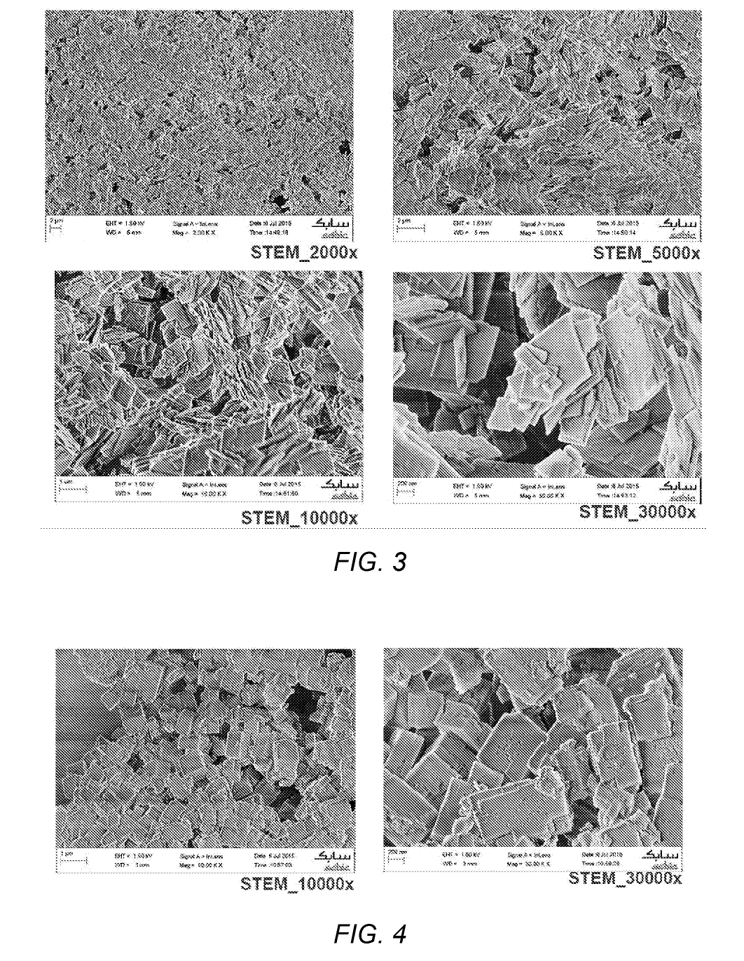

Catalyst 100 was characterized by scanning transmission electron microscopy (SEM). Catalyst 100 had a nano-platelet crystal morphology having thickness of less than 20 nm. FIG. 3 shows scanning transmission electron microscopy (STEM) images of the nano-platelet SAPO-34 molecular sieve at 2000×, 5000×, 10000×, and 30000× magnifications.

Comparative Catalyst 200, Conventional SAPO-34:

Comparative catalyst 200 was characterized using SEM. Comparative catalyst 200 had a cubic crystal morphology having 0.2 to 4 micron diameters. FIG. 4 shows scanning transmission electron microscopy (STEM) images of the conventionally prepared SAPO-34 molecular sieve at 10000× and 30000× magnifications.

### Example 4

### Methyl Chloride Conversion Reactions of Catalyst 100 and Comparative Catalyst 200

Catalysts 100 and comparative catalyst 200 were tested for methyl chloride conversion by using a fixed-bed tubular reactor at about 450° C. for a period of 5 h. For catalytic testing the powder catalysts were pressed and then crushed and sized between 20 and 40 mesh screens. In each test a fresh load of sized (20-40 mesh) catalyst (1.0 g) was loaded in a stainless steel tubular (½-inch outer diameter) reactor. The catalyst was dried at 200° C. under N₂ flow (100 cm³/min) for an hour and then temperature was raised to 450° C. at which time N₂ was replaced by methyl chloride feed (100 cm³/min) containing 20 mol % CH₃Cl in N₂. The weight hourly space velocity (WHSV) of CH₃Cl was about 0.8 h⁻¹ to 3.0 h⁻¹ and reactor inlet pressure was about 0 MPa. The SAR, percent CH₃Cl conversion, turn over frequency (TOF), C₂ percent selectivity, C₃ percent selectivity of the catalysts of present invention are listed in Table 2. Selectivities were based on methyl chloride and are carbon-based.

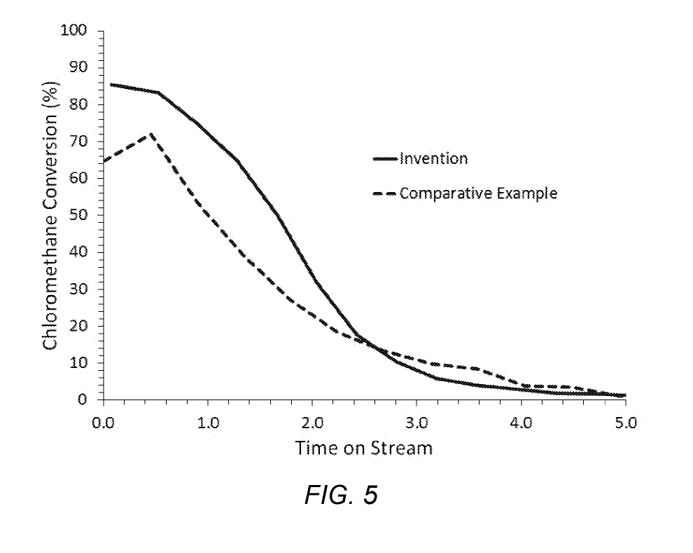

Table 2 below provides the CH₃Cl conversion and selectivity to C₂ and C₃ olefins at 5 h run time for the catalyst 100 and 200. FIG. 5 shows graphical data for chloromethane conversion comparing the nano-platelet SAPO-34 catalyst 100 with a comparative catalyst 200 prepared SAPO-34 molecular sieve 200.

TABLE 2

| | Conversion | | C₂ Olefin | C₃ Olefin | |
|---|---|---|---|---|---|
| Catalyst | (%) | TOF | Selectivity | Selectivity | STY* |
| 100 | 64 | 1.94 | 54 | 32 | 1.10 |
| 200 | 38 | 1.16 | 53 | 33 | 0.67 |

*Space Time Yield (Tonnes [C₂ + C₃]/Tonnes Catalyst/hr)

As shown in FIG. 5 and Table 2, the nano-platelet SAPO-34 catalyst had a higher initial chloromethane conversion.

1. A silicoaluminophosphate (SAPO)-34 molecular sieve comprising SiO₂/Al₂O₃/P₂O₅, wherein the SAPO-34 molecular sieve has platelet morphology and the thickness of the platelet is less than 20 nm.

2. The SAPO-34 molecular sieve of claim 1, wherein the synthesis molar ratio of SiO₂/Al₂O₃/P₂O₅ is 0.6:1:1.

3. The SAPO-34 molecular sieve of claim 1, wherein the SAPO-34 molecular sieve has been templated from a tetraethylammonium hydroxide (TEAOH) under hydrothermal conditions.

4. The SAPO-34 molecular sieve of claim 1, wherein the SAPO-34 molecular sieve does not have a spherical morphology, a flower morphology, or a cubic morphology.

5. The SAPO-34 molecular sieve of claim 1, wherein the SAPO-34 molecular sieve is capable of converting an alkyl halide to an olefin.

6. The SAPO-34 molecular sieve of claim 5, wherein the SAPO-34 molecular sieve has greater alkyl halide conversion as compared to 0.2 to 4 micron SAPO-34 molecular sieve cubic particles.

7. The SAPO-34 molecular sieve of claim 6, wherein the SAPO-34 molecular sieve initial alkyl halide conversion is least 80%.

8. A method for converting an alkyl halide to an olefin, the method comprising contacting a SAPO-34 catalyst with a feed comprising an alkyl halide under reaction conditions sufficient to produce an olefin hydrocarbon product comprising C₂-C₄ olefins, wherein the SAPO-34 molecular sieve catalyst has a platelet morphology and has been templated under hydrothermal conditions from a mixture comprising a quaternary ammonium salt.

9. The method of claim 8, wherein the quaternary ammonium salt is tetraethylammonium hydroxide (TEOH).

10. The method of claim 8, wherein the smallest dimension of the platelet is 20 nm.

11. The method of claim 8, wherein the alkyl halide is methyl chloride, methyl bromide, methyl fluoride, or methyl iodide, or any combination thereof.

12. The method of claim 8, wherein the molecular sieve has greater alkyl halide conversion as compared to 0.2 to 4 micron SAPO-34 molecular sieve cubic particles.

13. The method of claim 12, wherein the maximum selectivity of ethylene is 50% to 60% and the maximum selectivity of propylene is 35% to 45%. wherein the maximum combined selectivity of ethylene and propylene is at least 70%, wherein the maximum combined space time yield of ethylene and propylene is at least 1/hr or 1/hr to 3/hr, and/or wherein the maximum conversion of alkyl halide is at least 65% or 70% to 80%.

14. The method of claim 8, wherein the reaction conditions include a temperature from 300° C. to 500° C., a pressure of 5 atm or less, and a weighted hourly space velocity (WHSV) of 0.5 to 10 h⁻¹.

15. A method for preparing a silicoaluminophosphate (SAPO)-34 molecular sieve of claim 1, the method comprising:
(a) obtaining a mixture comprising water, an aluminum source, a silicon source, a phosphorous source, and a templating agent, wherein the templating agent comprises a quaternary ammonium salt;
(b) treating the mixture to a temperature of 150° C. to 170° C. under autogenous pressure to obtain a crystalline material; and
(c) removing the templating agent from the crystalline material to obtain the SAPO-34 molecular sieve.

16. The method of claim 15, wherein the mixture in (a) has a molar composition of:
aR:bSiO₂:cAl₂O₃:dP₂O₅:eH₂O,
where R is the templating agent, and a is 0-1, b is 0-1, c is 0-1, d is 0-1, and e is 30 to 80.

17. The method of claim 16, wherein a is 1, b is 0.65, c is 1, d is 1, and e is 45.

18. The method claim 15, wherein the aluminum source is aluminum isopropoxide, the phosphorous source is phosphoric acid, and the silicon source is colloidal silica, fumed silica, or tetraethyl orthosilicate.

19. The method of claim 15, wherein the templating agent further comprises tetraethylammonium hydroxide.

20. The method of claim 15, wherein step (b) is performed at a temperature of 140° C. to 160° C., step (c) comprises subjecting the crystalline material to a temperature of 500° C. to 600° C. for 3 hours to 10 hours, or both.
